(19) United States
(12) Patent Application Publication
(10) Pub. No.: US 2007/0141636 A1
(43) Pub. Date: Jun. 21, 2007

(54) Method for identifying androgen receptor modulators with full or mixed agonist activity

(76) Inventor: Fang Chen, North Wales, PA (US)

Correspondence Address: MERCK AND CO., INC, P O BOX 2000, RAHWAY, NJ, 07065-0907, US

(21) Appl. No.: 10/580,285

(22) PCT Filed: Nov. 19, 2004
(86) PCT No.: PCT/US04/38859

§ 371 Date: May 24, 2006

Related U.S. Application Data
(60) Provisional application No. 60/524,455, filed on Nov. 24, 2003.

(51) Int. Cl. G01N 33/567

(52) U.S. Cl. 435/7.2

(57) ABSTRACT

A method for identifying androgen receptor (AR) modulators which have full AR agonist or antagonist activity or mixed AR agonist activity is described. In one aspect of the invention, an analyte with mixed AR agonist activity is identified by comparing binding of the analyte to a human AR to its binding to the ligand binding domain (LBD) of the rhesus monkey AR (or other non-human AR) wherein a difference in binding indicates the analyte likely has mixed agonist activity. In a further aspect of the method, the ratio of AR agonist to AR antagonist activity of the analyte with the mixed AR agonist activity is determined by the effect of the analyte on PSA production in a prostrate cancer cell line in the presence and absence of a known full AR agonist. In a further still aspect of the method, analytes which have full AR agonist or antagonist activity are identified by the effect of the analyte on PSA production in a prostrate cancer cell line in the presence and absence of a known full AR agonist. The present invention is particularly useful for identifying analytes which are selective androgen receptor modulators (SARMs).

### BACKGROUND OF THE INVENTION

(1) Field of the Invention

The present invention relates to a method for identifying androgen receptor (AR) modulators which have full AR agonist or antagonist activity or mixed AR agonist activity. In one aspect of the invention, an analyte with mixed AR agonist activity is identified by comparing binding of the analyte to a human AR to its binding to the ligand binding domain (LBD) of the rhesus monkey AR (or other non-human AR) wherein a difference in binding indicates the analyte likely has mixed agonist activity. In a further aspect of the method, the ratio of AR agonist to AR antagonist activity of the analyte with the mixed AR agonist activity is determined by the effect of the analyte on PSA production in a prostrate cancer cell line in the presence and absence of a known full AR agonist. In a further still aspect of the method, analytes which have full AR agonist or antagonist activity are identified by the effect of the analyte on PSA production in a prostrate cancer cell line in the presence and absence of a known full AR agonist. The present invention is particularly useful for identifying analytes which are selective androgen receptor modulators (SARMs).

(2) Description of Related Art

The androgen receptor (AR) is a member of the nuclear receptor superfamily, which has been characterized as ligand-inducible transcription factors. The superfamily is composed of a group of structurally related receptors regulated by chemically distinct ligands. A commonly shared structure for the nuclear receptors is the highly conserved DNA binding domain (DBD) located in the center of the peptide, as well as the conserved ligand-binding domain (LBD) at C-terminus. Receptors of the same gene from different species usually have almost identical DBDs and core LBD domains. They exhibit the same binding affinity to the same ligand when expressed under the same condition using either full-length AR or LBD only.

The AR is expressed in numerous tissues of the body and is the receptor through which the physiological as well as the pathophysiological effects of endogenous androgen ligands such as testosterone (T), 5-α-dihydrotestosterone (DHT), and the like carry out their actions. Structurally, the AR is composed of three main functional domains: the ligand binding domain (LBD), the DNA-binding domain (DBD), and amino-terminal domain. A compound that binds to the AR and mimics the effects of an endogenous AR ligand is referred to as an AR agonist, whereas a compound that inhibits the effects of an endogenous AR ligand is termed an AR antagonist.

Ligands which can bind the LBD of the AR include pure or full agonists, pure or full antagonists, and mixed or partial agonists. Ligands with agonist activity bind the LBD of the AR which puts the AR in an active mode. In the active mode, the AR binds to its response elements in the regulatory sequences of one or more target genes which turns on or up-regulates expression of the one or more target genes. Ligands with antagonist activity also bind the LBD of the AR; however, the binding usually locks the AR in an inactive mode which has the effect of inhibiting AR transcriptional activity inducible by other agonists. Ligands with mixed agonist activity act as either agonists or antagonists depending on binding conditions such as tissue type, receptor density, coupling efficiency to effector molecules, and the like. For example, at high concentrations, ligands with mixed agonist activity can reduce other agonist activity.

In the absence of bound ligand, the receptor is in the form of an apo-receptor complex which includes HSP90, HSP70, HSP40, and other molecular chaperones. During synthesis, chaperones facilitate receptor folding into a high affinity ligand binding conformation and they keep the receptor in the high affinity conformation after synthesis (Caplan et al., J. Biol. Chem. 270: 5251-5257 (1995); Fliss et al., J. Biol. Chem. 274:. 34045-34052 (1999); Fang et al., J. Biol. Chem. 271: 28697-28702 (1996)). The AR is related to the glucocorticoid receptor (GR) and the estrogen receptor (ER). While the GR and the ER are relatively stable when not bound to ligand, the AR is rapidly turned over when not bound to ligand (Kemppainen et al., Biol. Chem 267: 968-974 (1992),). Chaperones may play an important role in maintaining the stability and high ligand binding affinity of the AR.

A number of natural or synthetic AR agonists have been clinically investigated for the treatment of musculoskeletal disorders such as bone disease(e.g., osteopenia and osteoporosis), bone formation, hematopoietic disorders (e.g., aplastic anemia and myelodysplastic syndromes), neuromuscular disease, neurodegenerative diseases such as Alzheimer's, cognitive impairment, rheumatological disease, wasting disease, obesity, sexual dysfunction, male disorders such as reproductive disorders and primary or secondary male hypogonadism, and for hormone replacement therapy (HRT), such as female androgen deficiency. In addition, AR antagonists, such as flutamide and bicalutamide, have been used to treat prostate cancer.

Because AR receptors are found in a wide variety of tissue types and cells and many diseases are specific to particular tissues or cells, it would be desirable to have AR agonists or antagonists that either activate (agonize) or deactivate (antagonize) the function of the AR in a target tissue or cell type which would afford the desired effect in the target tissue without exerting an effect in non-target tissues. Thus, mixed or partial AR agonists are particularly desirable because they can have different effects depending on the tissue or cell type.

Based on our experiments, SARMs (Selective Androgen Receptor Modulators) exhibit mixed actions in vivo which have both agonist and antagonist activity on the AR depending on the specific target tissue. Because SARMs are tissue selective, they can be used in treatments in which beneficial or desired effects in the target tissue are maintained while negative effects in non-target tissues are avoided. For example, SARMs that separate osteoanabolism from virilization would provide an acceptable profile for use in preventing fractures in the elderly. In general, identifying SARMS (mixed agonists) is a time consuming process. Therefore, there is a need for a method to predict SARMs compounds which would be useful for the treatment of diseases or disorders in which the AR is implicated.

### BRIEF SUMMARY OF THE INVENTION

The present invention provides a method for identifying androgen receptor (AR) modulators which have full AR agonist or antagonist activity or mixed AR agonist activity. In one aspect of the invention, an analyte with mixed AR agonist activity is identified by comparing binding of the analyte to a human AR to its binding to the ligand binding domain (LBD) of the rhesus monkey AR (or other non-human AR) wherein a difference in binding indicates the analyte likely has mixed agonist activity. In a further aspect of the method, the ratio of AR agonist to AR antagonist activity of the analyte with the mixed AR agonist activity is determined by the effect of the analyte on PSA production in a prostrate cancer cell line in the presence and absence of a known full AR agonist. In a further still aspect of the method, analytes which have full AR agonist or antagonist activity are identified by the effect of the analyte on PSA production in a prostrate cancer cell line in the presence and absence of a known full AR agonist. The present invention is particularly useful for identifying analytes which are selective androgen receptor modulators (SARMs).

Therefore, in one embodiment, the present invention provides a method for determining whether an analyte has a mixed androgen receptor (AR) agonist activity or an AR activity selected from the group consisting of full AR agonist activity and full AR antagonist activity, which comprises (a) providing a full-length human AR and an AR ligand binding domain polypeptide (ARLBD); (b) mixing the full-length human AR with the analyte and a labeled agonist in a first binding reaction and determining a first 50% inhibitory concentration (IC50) of the analyte for the full-length human AR; (c) mixing the ARLBD with the analyte and the labeled agonist in a second binding reaction and determining a second IC50 of the analyte for the ARLBD; and (d) comparing the first IC50 to the second IC50 wherein a first IC50 which is substantially the same as a second IC50 determines that the analyte has the AR activity selected from the group consisting of the full AR agonist activity and the full AR antagonist activity and wherein a second IC50 which is less than the first IC50 determines that the analyte has the mixed AR agonist activity.

In a further embodiment, the full-length human AR and the ARLBD are each provided in a cell extract. In a further still embodiment, the full-length human AR is provided as an extract from cells which express the full-length human AR. Preferably, the full-length human AR is endogenously expressed in human cells such as the MDA-MB453 cells which have been deposited as ATCC HTB-131. In further still embodiments, the ARLBD is provided as an extract from recombinant yeast cells such as Schizosaccaromyces pombe and Saccharomyces cerevisiea transfected with a vector which expresses the ARLBD, mammalian cells such as COS cells and the like transiently or stably transfected with a vector which expresses the ARLB, or bacterial cells such as BL21 cells transformed with a vector which expresses the ARLBD, or insect cells transfected with a baculovirus vector which expresses the ARLBD. In further still embodiments, the ARLBD is a non-human ARLBD selected from the group consisting of rat ARLBD, mouse ARLBD, or rhesus monkey ARLBD.

In further embodiments, the first and second binding reactions are hydroxyapatite-based displacement reactions. In further still embodiments, the labeled agonist is selected from the group consisting of methyltrienolone (R1881) and 5-α-dihydrotestosterone (DHT). In further still embodiments, the agonist is selected from the group consisting of R1881 and DHT and an IC50 is determined for the agonist. In other embodiments, in step (b) of the method the analyte is labeled and the agonist is unlabeled.

In further still embodiments, the analyte is determined to have the mixed agonist activity when the second IC50 is about five-fold less than the first IC50.

In further embodiments, the method includes a control binding assay wherein the full-length human AR and the ARLBD are each mixed with an agonist or an antagonist and an IC50 is determined for the agonist or the antagonist.

The present invention further provides a method for determining whether an analyte has an AR selected from the group consisting of full AR agonist activity, a full AR antagonist activity, and mixed AR agonist activity, which comprises:

(a) providing an analyte which has been determined to have the AR activity selected from the group consisting of full AR agonist activity, full AR antagonist activity, and mixed agonist activity according to the method of any one of Claims 1-12; (b) providing prostrate tumor cells which produce prostrate-specific tumor antigen (PSA) in the presence of an AR agonist; (c) adding the prostrate tumor cells to a medium which includes an agonist to provide a first culture of the cells and adding the prostrate tumor cells to a medium without the agonist to provide a second culture of the cells; (d) adding the analyte of step (a) to the first culture and the second culture and incubating the cells with the analyte for a time sufficient for a control culture of the cells, which includes the agonist and not the analyte, to produce the PSA; and (e) detecting the amount of PSA produced by the cells in the first culture and the second culture wherein (i) the analyte which stimulates the cells in the second culture to produce the PSA and further stimulates cells in the first culture to produce the PSA determines the analyte to have the full AR agonist activity, (ii) the analyte which does not stimulate the cells in the second culture to produce the PSA and which reduces the amount of the PSA produced by the cells in the first culture determines the analyte to have the full AR antagonist activity, and (iii) the analyte stimulates the cell in the second culture to produce the PSA and antagonizes the ability of the agonist in the first culture to stimulate the production of the PSA determines the analyte to have the mixed agonist activity.

In a further embodiment, the prostrate tumor cells are LnCap cells which have been deposited as ATCC CRL-1740 or CRL-1740D. In a further still embodiment, the amount of the PSA is detected by ELISA. In further still embodiments, the agonist is selected from the group consisting of R1881 and DHT.

The present invention further provides a method for determining the ratio of agonist to antagonist activity of an analyte having mixed AR agonist activity, which comprises (a) providing an analyte which has been determined to have the mixed AR agonist activity according to the method as set forth above; (b) providing prostrate tumor cells which produce PSA in the presence of an AR agonist; (c) adding the prostrate tumor cells to a medium which includes an agonist to provide a first culture of the cells and adding the prostrate tumor cells to a medium without the agonist to provide a second culture of the cells; (d) adding the analyte of step (a) to the first culture and the second culture and incubating the cells with the analyte for a time sufficient for a control culture of the cells, which includes the agonist and not the analyte, to produce the PSA; and (e) detecting the amount of the PSA produced by the cells in the second culture and the first culture wherein the amount of PSA produced in the second culture and the amount of PSA produced in the first culture determines the ratio of agonist to antagonist activity of the analyte.

In a further embodiment, the prostrate tumor cells are LnCap cells which have been deposited as ATCC CRL-1740 or CRL-1740D. In a further still embodiment, the amount of the PSA is detected by ELISA. In further still embodiments, the agonist is selected from the group consisting of R1881 and DHT.

The present invention further provides a method for identifying an analyte which has mixed AR agonist activity, which comprises: (a) providing a full-length human AR and an ARLBD; (b) mixing the full-length human AR with the analyte and a labeled agonist in a first binding reaction and determining a first 50% inhibitory concentration (IC50) of the analyte for the full-length human AR; (c) mixing the ARLBD with the analyte and the labeled agonist in a second binding reaction and determining a second IC50 of the analyte for the ARLBD; and (d) comparing the first IC50 to the second IC50 wherein a wherein a second IC50 which is less than the first IC50 identifies the analyte as having the mixed AR agonist activity.

In a further embodiment, the full-length human AR and the ARLBD are each provided in a cell extract. In a further still embodiment, the full-length human AR is provided as an extract from cells which express the full-length human AR. Preferably, the full-length human AR is endogenously expressed in human cells such as the MDA-MB453 cells which have been deposited as ATCC HTB-131. In further still embodiments, the ARLBD is provided as an extract from recombinant yeast cells such as Schizosaccaromyces pombe and Saccharomyces cerevisiea transfected with a vector which expresses the ARLBD, mammalian cells such as COS cells and the like transiently or stably transfected with a vector which expresses the ARLB, or bacterial cells such as BL21 cells transformed with a vector which expresses the ARLBD. In further still embodiments, the ARLBD is a non-human ARLBD selected from the group consisting of rat ARLBD, mouse ARLBD, or rhesus monkey ARLBD.

In further embodiments, the first and second binding reactions are hydroxyapatite-based displacement reactions. In further still embodiments, the labeled agonist is selected from the group consisting of R1881 and DHT. In further still embodiments, the agonist is selected from the group consisting of R1881 and DHT and an IC50 is determined for the agonist. In other embodiments, in step (b) of the method the analyte is labeled and the agonist is unlabeled.

In further still embodiments, the analyte is determined to have the mixed agonist activity when the second IC50 is about five-fold less than the first IC50.

In further embodiments, the method includes a control binding assay wherein the full-length human AR and the ARLBD are each mixed with an agonist or an antagonist and an IC50 is determined for the agonist or the antagonist.

In a further embodiment, the above method further includes determining the ratio of agonist to antagonist activity of the analyte by (i) providing prostrate tumor cells which produce PSA in the presence of an AR agonist; (ii) adding the prostrate tumor cells to a medium which includes an agonist to provide a first culture of the cells and adding the prostrate tumor cells to a medium without the agonist to provide a second culture of the cells; (iii) adding the analyte to the first culture and the second culture and incubating the cells with the analyte for a time sufficient for a control culture of the cells, which includes the agonist and not the analyte, to produce the PSA; and (iv) detecting the amount of the PSA produced by the cells in the second culture and the first culture wherein the amount of PSA produced in the second culture and the amount of PSA produced in the first culture determines the ratio of agonist to antagonist activity of the analyte.

In a further embodiment, the prostrate tumor cells are LnCap cells which have been deposited as ATCC CRL-1740 or CRL-1740D. In a further still embodiment, the amount of the PSA is detected by ELISA. In further still embodiments, the agonist is selected from the group consisting of R1881 and DHT.

The term “full agonist” refers to a molecule that binds to a receptor to form a complex which elicits a full pharmacological response, peculiar to the nature of the receptor involved. For example, a hormone acts as an agonist when it binds to its receptor and triggers a biochemical response like the endogenous hormone.

The term “full antagonist” refers to a molecule that binds to a receptor to form a complex which does not give rise to a pharmacological response. It also inhibited endogenous hormone activity. The term further includes molecules which partially or completely inhibit the effect of another molecule. For example, a molecule that blocks a hormone's receptor site is the hormone's antagonist. The term “antagonistic effect” refers to the consequence of one molecule or group of molecules counteracting the effects of another molecule; the opposing molecules cancel out each other's effects. Typically, the effect of an antagonist is observed as a blocking of activation by an agonist. Antagonists include competitive and non-competitive antagonists. A competitive antagonist (or competitive blocker) interacts with or near the site specific for the agonist (for example, a ligand or neurotransmitter) for the same or closely situated site. A non-competitive antagonist or blocker inactivates the functioning of the receptor by interacting with a site other than the site that interacts with the agonist.

The term “mixed agonist” or “partial agonist” refers to a molecule which possesses affinity for a receptor, but unlike a full agonist, will elicit only a small degree of the pharmacological response peculiar to the nature of the receptor involved, even if a high proportion of receptors are occupied by the compound. Thus, a mixed agonist is useful for treatments where particular receptors or receptors in particular tissues are to be targeted by the mixed agonist.

R1881 (or methyltrienolone) is a synthetic non-aromatizable androgen and anabolic steroid. It is an AR agonist that binds strongly to the AR and, therefore, has been used as an affinity label for the AR in the prostate and in prostatic tumors. The chemical formula for R1881 is 17 β-hydroxy-17-methyl-estra-4,9,11-trien-3-one. R1881 is a full AR agonist which is slightly less potent than DHT and does not antagonize 0.5 nM R1881 or DHT activity.

DHT (5-α-dihydrotestosterone) is a naturally occurring AR agonist that binds strongly to the AR. DHT is a full AR agonist; it displays full agonist activity on its own and in the presence of 0.5 nM DHT or R1881.

### BRIEF DESCRIPTION OF THE DRAWINGS

FIG. 1 shows the chemical structures of several natural and synthetic AR ligands.

FIG. 2A shows a comparison of the IC50s for AR full agonist R1881 for full-length human AR (mdaAR) and to rhARLBD. As shown, DHT has the same binding affinity to full-length human AR and to rhARLBD.

FIG. 2B shows a comparison of the IC50s for AR full agonist DHT for full-length human AR and to rhARLBD. As shown, R1881 has the same binding affinity to full-length human AR and to rhARLBD.

FIG. 2C shows a comparison of the IC50s for AR full antagonist CASODEX for full-length human AR and to rhARLBD. As shown, CASODEX has the same binding affinity to full-length human AR and to rhARLBD.

FIG. 2D shows a comparison of the IC50s for AR weak agonist dehydroepiandrosterone (DHEA) for full-length human AR and to rhARLBD. As shown, DHEA has reduced binding affinity to rhARLBD as compared to full-length human AR.

FIG. 3A shows a comparison of the IC50s for AR partial agonist 4-androstene-3β,17β-diol for full-length human AR and to rhARLBD. As shown, the 4-androstene-3β,17β-diol has reduced binding affinity to rhARLBD as compared to full-length human AR.

FIG. 3B shows a comparison of the IC50s for AR partial agonist 4-androstene-3,17β-dione for full-length human AR and to rhARLBD. As shown, 4-androstene-3,17β-dione has reduced binding affinity to rhARLBD as compared to full-length human AR.

FIG. 3C shows a comparison of the IC50s for AR partial agonist cyproterone acetate (CPA) for full-length human AR and to rhARLBD. As shown, CPA has reduced binding affinity to rhARLBD as compared to full-length human AR.

FIG. 3D shows a comparison of the IC50s for AR partial agonist Compound A for full-length human AR and to rhARLBD. As shown, Compound A has reduced binding affinity to rhARLBD as compared to full-length human AR.

FIG. 4A shows that DHT, R1881, 4-androstene-3,17β-dione, 4-androstene-3β,17β-diol, CPA, DHEA and Compound A all had different degree of agonist activity when each was separately administered to LnCap cells (in agonist mode). The Figure further shows that CASODEX is an antagonist. PSA levels were measured 24-48 hours after administering the compound and were compared to the levels produced with 10 nM DHT.

FIG. 4B shows that 4-androstene-3,17β-dione, 4-androstene-3β,17β-diol, CPA, DHEA, Compound A and Casodex all had a different degree of antagonist activity when each was separately administered to LnCap cells treated with 0.5 nM DHT or R1881 (antagonist mode). Percentage of inhibition=100×(Analyte−0.5 nM DHT or R1881)/0.5 nM DHT or R1881 of the corresponding PSA levels. Basal level is subtracted from each.

### DETAILED DESCRIPTION OF THE INVENTION

The present invention provides a novel method for identifying androgen receptor (AR) modulators which have either mixed AR agonist activity, full AR agonist activity, or full AR antagonist activity. The method involves comparing the binding of an analyte to the AR ligand binding domain (ARLBD) to its binding to a human AR. Analytes which bind to the ARLBD and the human AR with substantially similar affinity are Group I analytes. Group I analytes are AR modulators with either full agonist activity or full antagonist activity. Analytes which bind the ARLBD with significantly reduced affinity compared to its binding to the human AR are Group II analytes. Group II analytes are analytes which have different degrees of mixed agonist activity. In a further embodiment of the method, the ARLBD is provided as a recombinant protein comprising the ARLBD of the rhesus (Macaca mulatta) monkey or in further still embodiments, the ARLBD of the human AR or the ARLBD of a non-human species such as the rat or mouse. In particular embodiments, the human AR is a recombinant protein comprising a full-length human AR ectopically expressed in a human or non-human cell. Preferably, the full-length human AR is provided as a protein endogenously expressed in a human cell.

The present invention further provides an embodiment of the above method which enables the user to determine whether a Group I analyte is a full agonist or a full antagonist. In the second step, the analyte is administered to LnCap cells (a prostrate tumor cell line, ATCC CRL1740, 1740D) in culture as a mixture with a known full AR agonist such as DHT or R1881 (antagonist mode) or alone (agonist mode) and measuring expression of prostrate specific antigen (PSA) in both modes.

The present invention further still provides an embodiment of the above method which enables the user to determine the ratio of agonist to antagonist activity of an analyte identified as a Group II analyte by observing the effect of the analyte on PSA expression in LnCap cells in the agonist mode and the antagonist mode. Thus, the method is particularly useful for identifying analytes which can be candidates as selective androgen receptor modulators (SARMs).

Thus, the present invention is useful for identifying analytes which are useful for treating musculoskeletal disorders such as bone disease (e.g., osteopenia and osteoporosis), bone formation, hematopoietic disorders (e.g., aplastic anemia and myelodysplastic syndromes), neuromuscular disease, neurodegenerative diseases such as Alzheimer's, cognitive impairment, rheumatological disease, wasting disease, obesity, sexual dysfunction, male disorders such as reproductive disorders and primary or secondary male hypogonadism, and for hormone replacement therapy (HRT), such as female androgen deficiency.

The method of the present invention was discovered during a program to generate drug development reagents for bone formation in which AR was the target for osteo-anabolic ligand screening assays. The screening assays were set up to use the ARLBD from Macaca mulatta (rhesus monkey; herein after rhARLBD) expressed in yeast. The nucleic acids encoding the rhesus monkey AR and methods for expressing the rhesus monkey AR and rhARLBD in yeast have been previously disclosed in commonly owned WO02090529 to Towler and Chen. A medium to high throughput AR binding assay for screening analyte libraries and characterizing various analytes therein was also developed. The medium to high throughput AR binding assay was a hydroxyapatite (HAP)-based ³H-R1881 displacement binding assay.

The AR binding assay using the rhARLBD produced binding IC50s (inhibitory concentrations) needed to inhibit 50% of ³H-R1881 binding for AR full agonists R1881 and DHT of about 1 nM for each. This was the same as the IC50s which had been produced using full-length human AR. The result suggested that the rhARLBD could substitute for the full-length human AR in the screening assays. However, when SARMs compounds A and B were tested in the assay, the ICM50s using rhARLBD were unexpectedly about 15 fold or more higher than the IC50s using full-length human AR stably expressed in CHO cells (about 8 nM for B and about 30 nM for compound A). A further AR binding assay which used rat ARLBD in place of the rhARLBD produced an IC50 for compound A of about 600 nM, which was almost identical to the IC50 obtained using the rhARLBD. The unexpected greater than 15 fold increase in IC50s in assays which used rhARLBD or rat ARLBD over assays which used full-length human AR was consistently observed

To determine what caused the unexpected difference in IC50 values between the assays using human full-length AR and the assays using rhARLBD or rat ARLBD, the binding assay using the full-length human AR was repeated using human full-length AR expressed in MDA-MB453 cells. MDA-MB453 cells express high levels of endogenous human full-length AR and are available from the American Type Culture Collection (ATCC), P.O. Box 1549, Manassas, Va. 20108 as HTB-131. After the binding conditions using MDA-MB453 cell lysate had been established, the IC50s produced using full-length human AR endogenously expressed from the MDA-MB453 cells were the same as the IC50s produced using full-length human AR exogenously (ectopically) expressed in stably transfected CHO cells (about 8 nM for B and about 30 nM for compound A, for each). The comparison also revealed that dextran coated charcoal (DCC), hydroxyapatite (HAP), or proteinase inhibition were not the cause of the difference in IC50s between assays which used rhARLBD or rat ARLBD and assays which used full-length human AR. Therefore, it was concluded that the source of receptor might have been the reason for the difference in IC50s for compound A-like (SARM-like compound) using rhARLBD versus endogenous full-length human AR.

The results showed that while endogenously or stably transfected (ectopically) expressed human full-length AR retains its high binding affinity to compound A-like compounds, exogenously (ectopically) expressed rhARLBD, which is identical to human ARLBD in amino acid sequence, and rat ARLBD has reduced binding affinity to compound A-like (SARMs-like) compounds even though rhARLBD and rat ARLBD had the expected binding affinity to DHT or R1881. The above results suggested that LBD conformations are not identical between endogenous full-length ARs, transiently (ectopically) expressed full-length ARs, and transiently (ectopically) expressed ARLBD (rhesus monkey or rat). It appears that the chaperone composition necessary for stabilizing AR may not be optimal for transiently (ectopically) expressed ARLBD. SARM-like compounds are very sensitive to subtle changes of receptor conformation while DHT-like (full agonist) compounds are not. Thus, the unexpected results suggested that because of the differential binding of SARMs-like compounds to full-length human AR versus rhARLBD or rat ARLBD, an assay could be developed which would be able to identify and distinguish SARMs-like compounds (mixed or partial agonists) from full AR agonists and AR antagonists. In light of the sensitivity of SARMs-like compounds to AR conformation, cells or cell lines which endogenously express full-length human AR are preferred for use in the method of the present invention so as not to miss mixed agonist analytes such as SARM-like compounds. Such cells and cell lines include, but are not limited to, the MDA-MB453 cells were used as the AR receptor source in the primary binding assay.

LnCap is a human prostate tumor cell line available from the ATCC as CRL-1740 or CRL-1740D. Like MDA-MB-453 cells, LnCap cells also express high levels of endogenous full-length human AR. The cells are very responsive to androgen treatments in terms of cell proliferation and prostate-specific antigen (PSA) protein secretion. Even though there is only one mutation in AR from LnCap (at residue 877, threonine to alanine), this mutation has broadened the specificity of the AR. Estradiol, progesterone, and some anti-androgens (for example, cyproterone acetate (CPA), hydroxyflutamide, and the like) are able to stimulate cell growth through the AR. Receptor/reporter cotransfection assay using an LnCap AR expression vector showed that the residue 877 threonine to alanine mutation enables estradiol, progesterone, CPA, and hydroxyflutamide to have an agonist effect on the androgen promoter. However, the full antagonist CASODEX retains its antagonism on LnCap AR in LnCap growth stimulation or cotransfection assays. Experimental data has revealed that the antagonist identity of many mixed agonists, such as CPA, can be unmasked when treating LnCap cells in the presence of 0.5 nM DHT or R1881. As SARMs tend to be mixed agonists, an LnCap PSA production assay in agonist and antagonist modes was developed to further profile analytes that are classified in the above AR binding assays as full agonists or antagonists or mixed agonists.

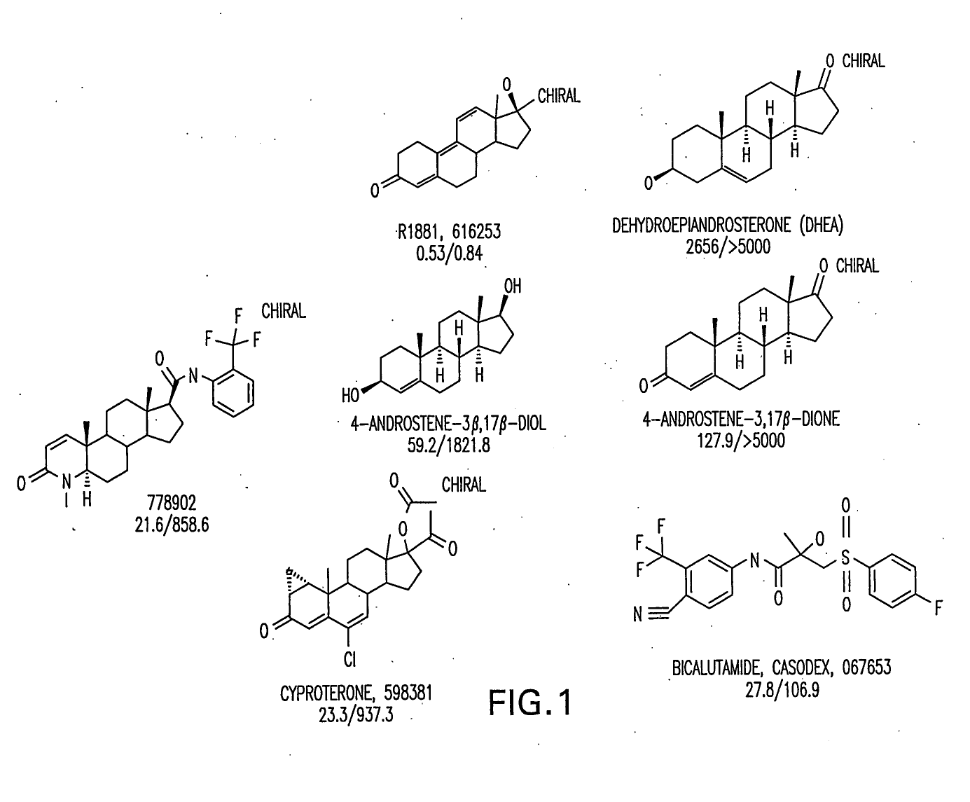

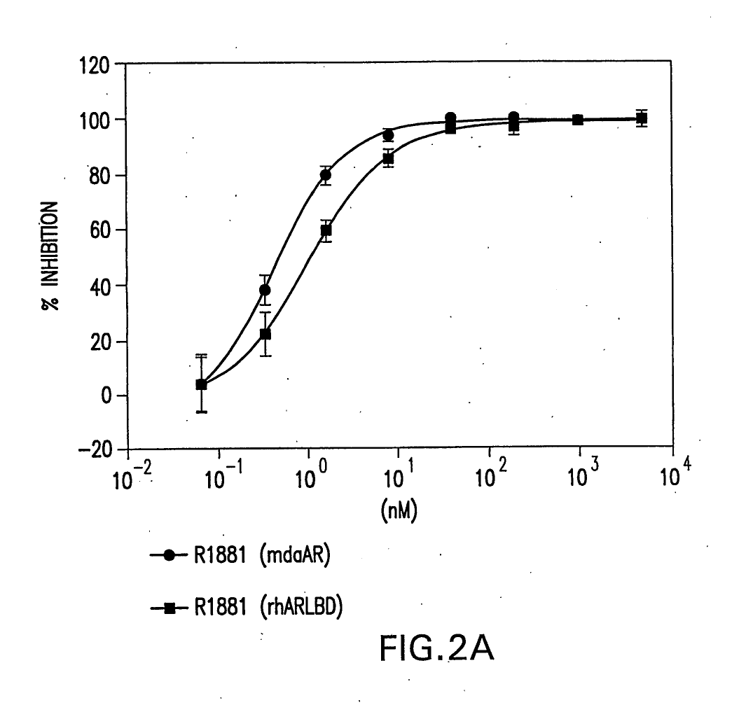

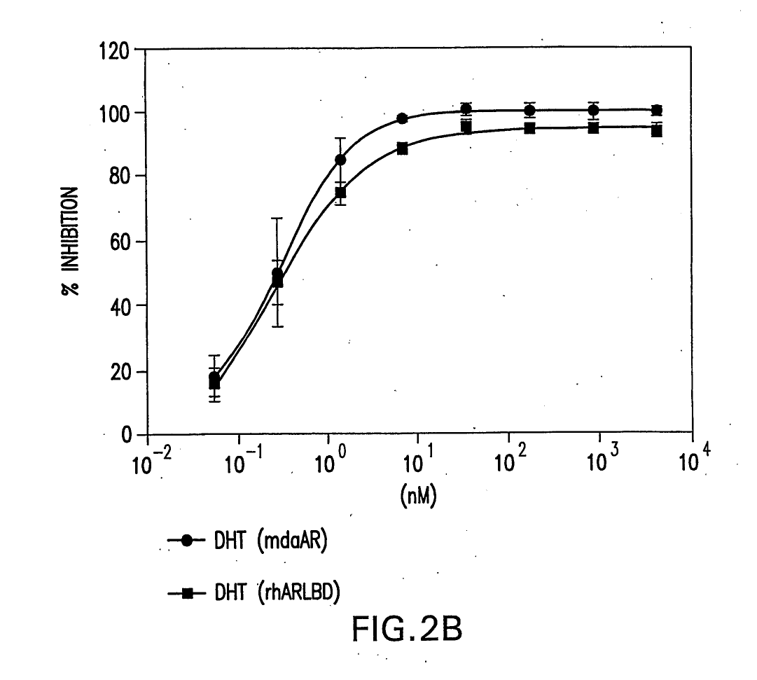

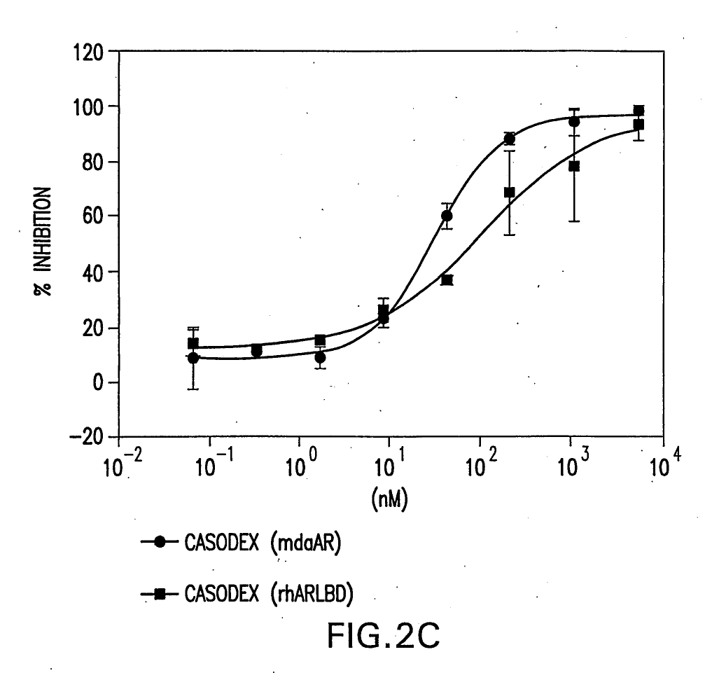

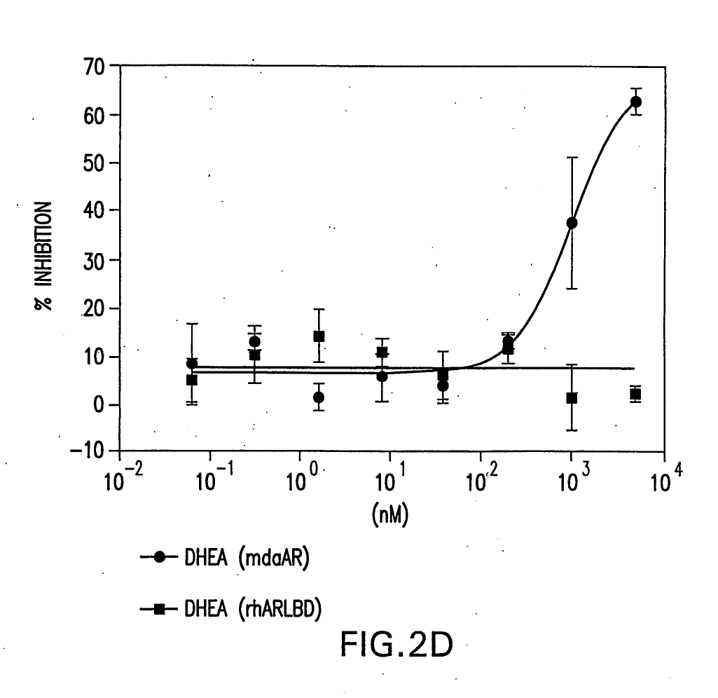

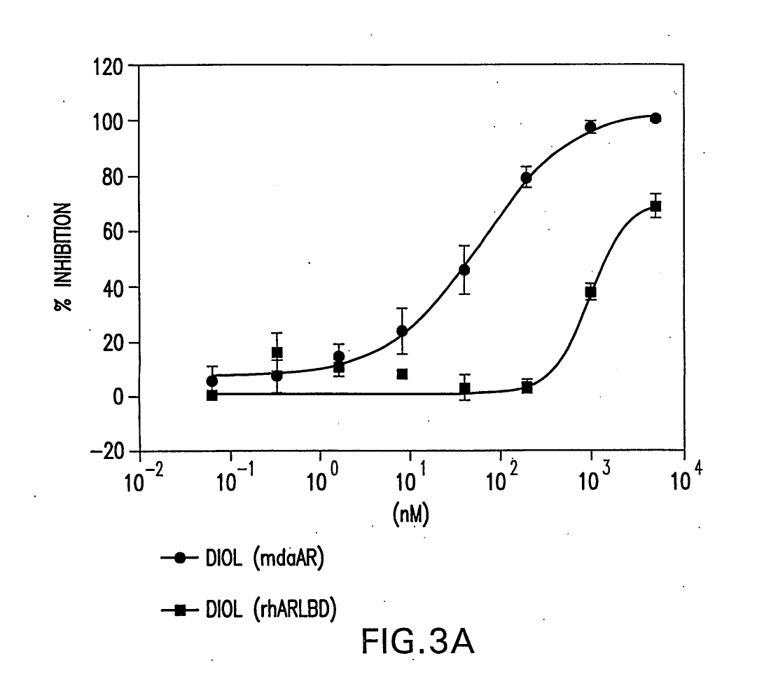

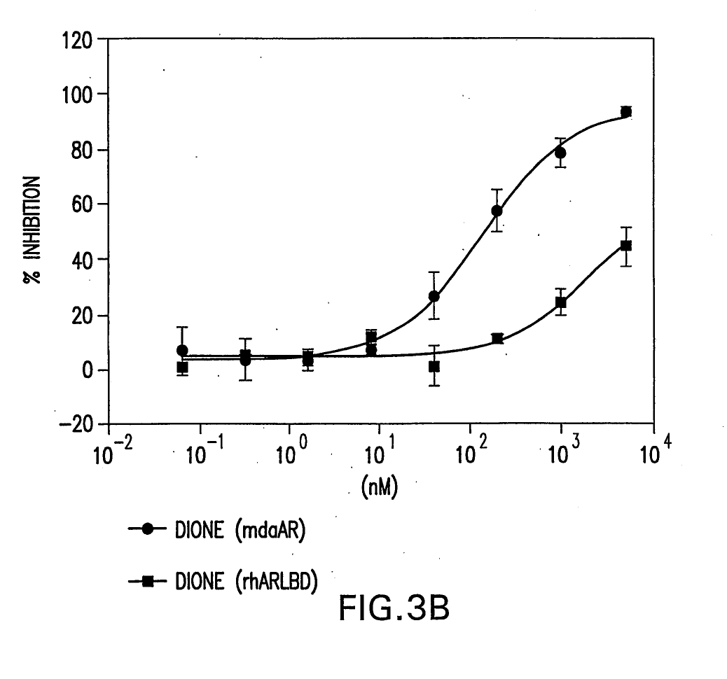

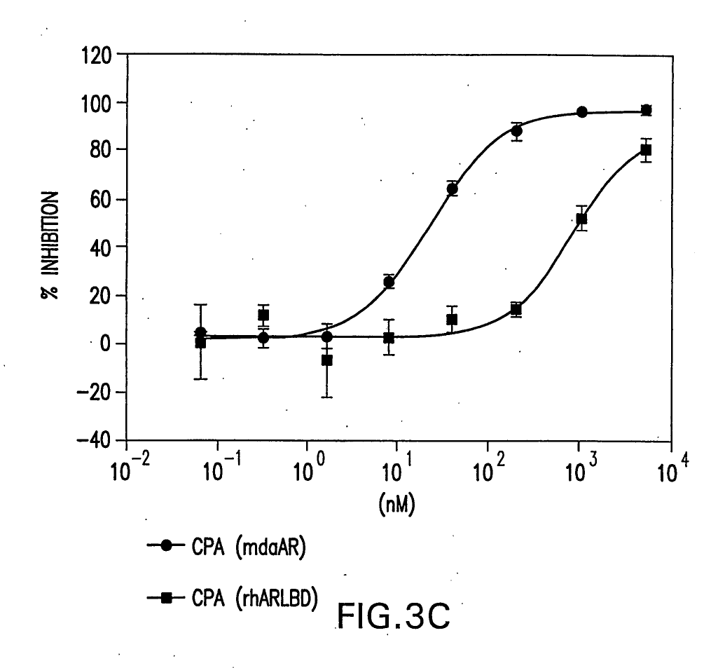

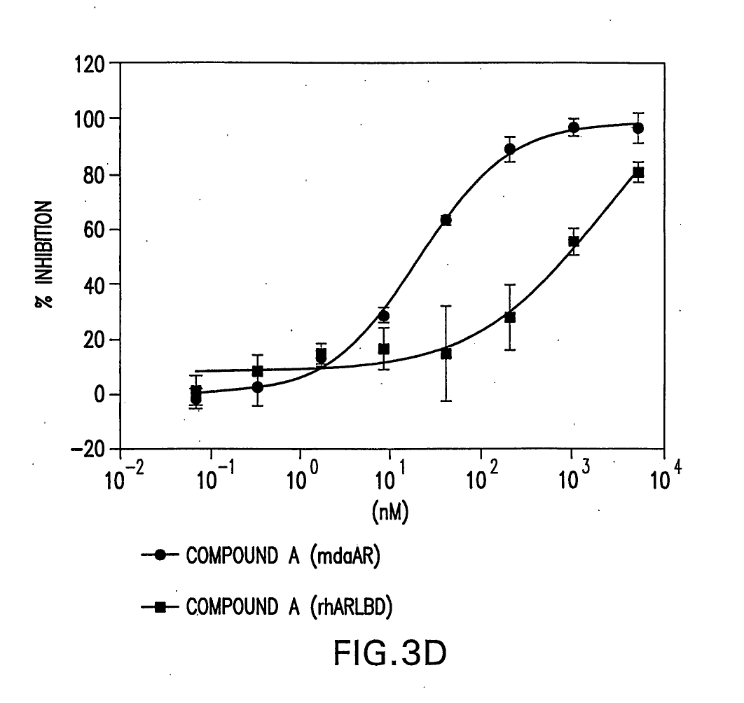

Experiments on a selected list of compounds including full agonists/antagonists and partial agonists were performed (See FIG. 1 for the compounds which were used). The binding assays using yeast lysates containing rhARLBD and MDA-MB453 cell lysate containing endogenously expressed full-length human AR showed that compounds of the list can be divided into two groups based on the binding data from the binding assays. One group of compounds maintained similar binding affinity to full-length human AR and rhARLBD (Group I). A similar binding affinity is where the binding difference is not significance, in general, a binding difference of less than five-fold is usually considered to be insignificant. This can be seen in FIGS. 2A-2D where there was no significant shift in binding between the full-length human AR and the rhARLBD for the full agonists DHT and R1881 or the full antagonist CASODEX. The other group of compounds exhibited high binding affinity to MDA hAR but with significantly reduced affinity to rhARLBD (Group II). A significantly reduced binding affinity is a binding affinity which is usually at least about five-fold less than the binding affinity to the full-length AR. This can be seen in FIGS. 3A-3D where there was a significant right shift (decrease in binding affinity) in binding between the full-length human AR and the rhARLBD.

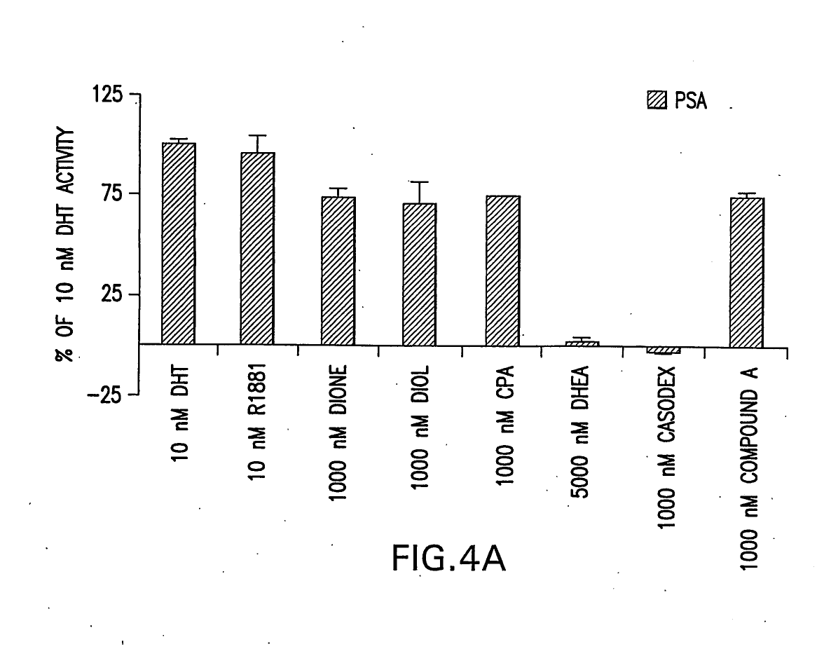

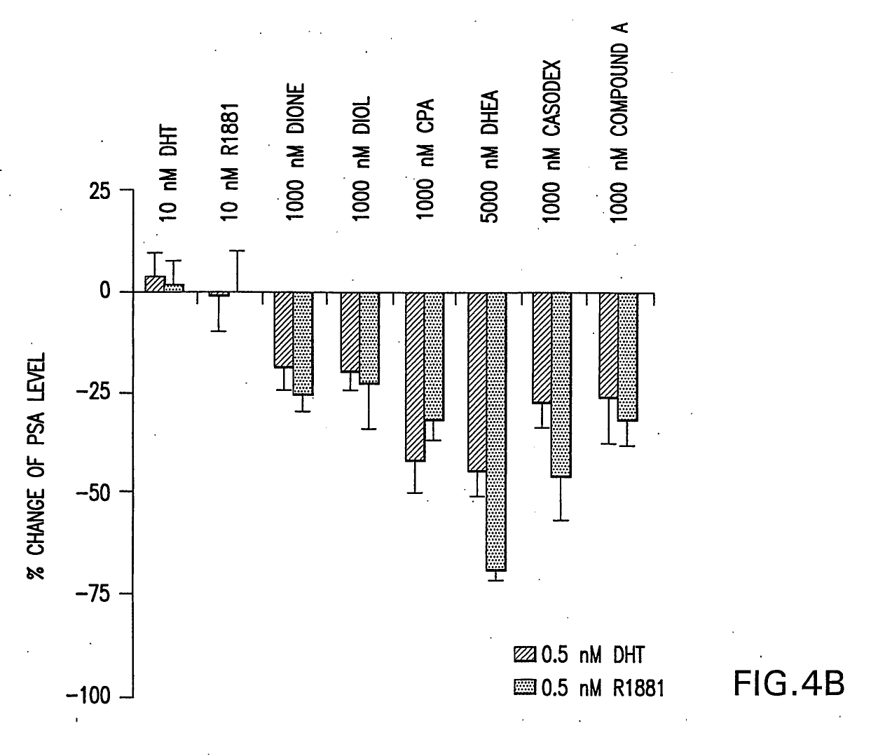

When the compounds were tested on LnCap cells in the absence (agonist mode) or presence of 0.5 nM R1881 or DHT (antagonist mode), the Group I compounds, which showed no significant shift in binding affinity, could be further distinguished as either full agonists or full antagonists. The full agonists stimulated PSA production in LnCap cells in the agonist mode and further stimulated PSA production in LnCap cells the presence of R1881 or DHT (antagonist mode). The full antagonists did not stimulate PSA production in LnCap cells in the agonist mode and reduced PSA production in LnCap cell in the antagonist mode. Those compounds that showed a significant right shift in binding rhARLBD compared to binding the full-length human AR and thus identified as mixed agonists (Group II) where further identified as mixed agonists in the LnCap PSA assays. They behaved like partial agonists in stimulating PSA production in LnCap cells in the agonist mode but reduced the PSA production in LnCap cells in the antagonist mode. It was further found that the relative ratio of agonist to antagonist activity of a mixed agonist was obtainable from the LnCap PSA assay. The results for the LnCap PSA assay are summarized in FIGS. 4A and 4B. The above assays demonstrated that SARM compound A exhibited mixed actions in LnCap PSA assays, just like their mixed behavior in in vivo assays.

In summary, the AR binding assays using lysates containing endogenously expressed full-length human AR and exogenously expressed rhARLBD can categorize compounds into two groups: Group I compounds with affinities to both forms of receptors which do not differ significantly and Group II compounds which have significantly different affinities for the two forms of receptors. Furthermore, LnCap PSA assays in agonist mode or antagonist mode can subdivide Group I compounds into full agonists or full antagonists and Group II compounds can be confirmed as being mixed agonists and can be further differentiated into groups of mixed agonist compounds based on the ratio of agonist activity to antagonist activity of the compound. The above compound profiling can assist in quick identification of SARM candidates which can be further tested in in vivo models.

Therefore, in light of the above, the present invention provides a method for identifying AR modulators which have either full AR agonist activity or full AR antagonist activity or mixed AR agonist activity. The method involves comparing the binding of an analyte to an ARLBD to its binding to a full-length human AR. Analytes which bind to the ARLBD and the full-length human AR with substantially similar affinity (no significant difference in affinity or an affinity which differs by less than about five-fold) are Group I analytes. Group I analytes are AR modulators with either full agonist activity or full antagonist activity. Analytes which bind the ARLBD with significantly reduced affinity compared to its binding to the human AR are Group II analytes. Group II analytes are analytes which have mixed agonist activity.

In an optional second step, the present invention further provides a method for determining whether a Group I analyte is a full agonist or a full antagonist and the relative ratio of agonist to antagonist activity of a Group II analyte. In the second step, the analyte is administered to LnCap cells or variant thereof or cells of similar characteristics in culture as a mixture with a known full agonist (antagonist mode) or alone (agonist mode) and measuring expression of prostrate specific antigen (PSA) in both modes. The ability of the method to identify mixed agonists and then to characterize the mixed agonists based on their relative ratio of agonist to antagonist activity renders the method particularly useful for identifying analytes which can be candidates of selective androgen receptor modulators (SARMs).

In a further embodiment of the method, the ARLBD is provided as a recombinant protein comprising the ARLBD of a human or an animal, preferably an animal such as a primate, rat, guinea pig, mouse, bovine, or the like. For example, the ARLBD can comprise the ARLBD of the rhesus monkey (Macaca mulatta) or the ARLBD rat or mouse. The ARLBD can be prepared and produced in a mammalian cell such as COS cells or the like, a bacterial cell such as BL21 or the like, plant cell or yeast cell such as Schizosaccaromyces pombe, Saccharomyces cerevisiea or the like, an insect cell such as Spodoptera frugiperda or the like using standard molecular biology methods. Molecular biology methods, including cloning methods which can be adapted to clone the rhARLBD, human ARLBD, rat ARLBD, and mouse ARLBD, and methods for expressing cloned proteins and portions thereof in cells are described in Sambrook et al., Molecular Cloning: A Laboratory Manual ₂nd Edition; Cold Spring Harbor Laboratory Press, Cold Spring Harbor, N.Y., (1989) or Sambrook and Russell, Molecular Cloning: A Laboratory Manual, 3rd Edition. Cold Spring Harbor Laboratory Press, Plainview, N.Y. (2001). The nucleotide sequence for the rhesus monkey AR can be found in WO02090529 to Towler and Chen, the nucleotide sequence for the human AR is available from L29496, the nucleotide sequence for the rat AR is available from GenBank NM_{—}012502, and mouse AR is available from GenBank NM_{—}013476. LBDs from other AR are believed to also be useful in the method disclosed herein. The ARLBD can be provided as a fusion protein in which the ARLBD is covalently joined to a non-ARLBD protein, e.g., glutathione S transferease (GST) or portion thereof, GAL4 protein or portion thereof, polyhistidine, and the like. In a preferred embodiment, the rhesus monkey AR is the source for the ARLBD. In further still embodiments, the full-length human AR is provided as protein, preferably as a protein ectopically or endogenously expressed in a human cell. In a preferred embodiment, the full-length human AR is endogenously expressed in MDA-MB453 cells or variant thereof or in any other cell line which endogenously expresses the full-length human AR.

A typical method is performed as follows. A human cell line which endogenously or ectopically expresses full-length human AR is cultured in culture medium for a time sufficient for the cells to produce the full-length human AR. The cells are harvested and lysed. For example, the cells can be suspended in a buffer of about neutral pH and lysed by freeze-thawing. Optionally, the cells can be lysed by homogenization or by using a French press or other means. The cell debris is removed and the remaining lysate, which contains the full-length human AR, is used for binding with an analyte. A transformed or transfected cell which expresses the ARLBD is cultured in a medium for a time sufficient to produce the ARLBD. The cells are harvested and lysed. For example, in the case of yeast cells, the cells are harvested and lysed by briefly vortexed in the presence of glass beads. The cell debris is removed and the remaining lysate, which contains the ARLBD, is used for binding with the analyte.

A displacement assay using various concentrations of analyte with aliquots of each AR receptor lysate in the presence of a labeled ligand (full agonist) is used to provide an IC50 for the analyte which is used to determine whether the analyte is a mixed agonist or a full agonist or full antagonist. Examples of ligands include R1881 or DHT. The ligand can be labeled by any standard technique known in the art, such as radiolabeling, fluorescence labeling, and the like. Labels can include radioisotopes, FITC or other fluorochrome markers, enzymes, biotin, digoxigenin, fluorogenic quencher-donor dyes, or other molecules capable of detection. For each concentration of analyte, an aliquot of the analyte is mixed in a first binding reaction with an aliquot of the lysate containing the ARLBD and an aliquot of the analyte is mixed in a second binding reaction with an aliquot of the lysate containing the full-length human AR, each binding reaction further containing the labeled ligand. In general, the binding reactions for each analyte comprises serial dilutions of the analyte or comprises a range of concentrations of the analyte. For example, a range of analyte concentrations can be from about 10⁻² nM to about 10⁴ nM. The amount of lysate used for each assay is based on titration using labeled-ligand at concentration about the Kd value of the ligand to the corresponding receptor. For example, a typical binding reaction can include about 50 to 100 μL of lysate and about 50 to 150 μL of binding buffer containing the analyte and labeled ligand. The binding reaction typically is at about pH 7.5 and is incubated at about 4° C. for a time sufficient for the labeled ligand added to the binding reaction to be substantially bound to the ARLBD or full-length human AR in the absence of the any analyte, in general about 18 hours. After about 18 hours, an equal volume of a slurry of hydroxyapatite (HAP) is added to each binding reaction to bind the analyte-receptor complexes or labeled ligand-receptor complexes. The binding reactions with HAP are incubated on ice for about 10 minutes to allow the analyte-bound receptor or ligand-bound receptor to bind the HAP, forming a HAP complex. The HAP complex is separated from unbound material (free analyte and labeled-receptor) by centrifugation or filtration. The HAP complex is washed and the label detected. The binding displacement results are compared to control assays using known ligands such as 5-alpha dihydrotestosterone and R1881. The amount of radioactivity bound to the HAP is inversely proportional to the binding affinity of the analyte for the ARLB or full-length human AR. The binding results from the full-length human AR and ARLBD are compared to identify analytes which have a significant reduction (right shift) in binding to the ARLBD compared to binding to the full-length human AR. In general, a right shift of about five-fold. That is, analytes which have less ability to displace the labeled ligand bound to the ARLBD than to displace labeled ligand bound to the full-length human AR (in other words, less ability to inhibit binding of the labeled ligand to the ARLBD compared to binding of the labeled ligand to the full-length human AR). Such analytes with reduced binding affinity for the ARLBD compared to their binding affinity for full-length human AR tend to be mixed agonists in the LnCap PSA assay.

In an alternative method, the analyte is labeled and the ligand is unlabeled. The assay is performed as above and the amount of radioactivity bound to the HAP is directly proportional to the binding affinity of the analyte for the ARLBD and full-length human AR. The binding results from the full-length human AR and ARLBD are compared like above to identify analytes which bind the ARLBD with substantially less affinity than they bind full-length human AR. Such analytes with reduced binding affinity for the ARLBD compared to their binding affinity for full-length human AR end to be mixed agonists in the LnCap PSA assay.

The second step distinguishes full agonists from full antagonists (Group I analytes), confirms the mixed agonists identified above are mixed agonists (Group II analytes), and determines the relative ratio of agonist to antagonist activity of the mixed agonists identified above. In general, the second step is performed as follows. LnCap cells are seeded into the wells of tissue culture plates. About four to 12 hours after seeding, for each well, an analyte is added in the absence of a known agonist (agonist mode) or in the presence of a known agonist (antagonist mode). In general, the amount of analyte which is added is that amount which results in about 100% inhibition of the binding of the labeled ligand to the full-length human AR. Examples of known agonists which are suitable for the assay are R1881 or DHT, either of which is preferably provided at a concentration of about 0.5 nM. In general PSA levels (or amounts) produced by the cells are measured about 24 to 48 hours later using a commercially available ELISA kit for detecting PSA (for example, the PSA ELISA kit available from DSL, Webster, Tex.). For the agonist mode, the PSA levels (or amounts) of produced by cells in the presence of the analyte are compared to the levels (or amounts) produced by a known agonist such as DHT or R1881, preferably in an amount of about 0.5 to 10 nM. For the antagonist mode, the relative decrease or inhibition of PSA production effected by the known agonist is determined by dividing the amount or level of PSA produced in the presence of the analyte and known agonist less the level or amount of PSA produced in the presence of the known agonist alone by the level or amount of PSA produced in the presence of the known agonist alone 100×(PSA_{analyte}-PSA_{known agonist})/PSA_{known agonist}].

In the agonist mode, full agonists and mixed agonists effect an increase in PSA levels (or amounts) whereas full antagonists have no significant effect on PSA levels (or amounts) or effect a decrease in PSA levels (or amounts). In the antagonist mode, full agonists effect a further increase in PSA levels (or amounts) over the level (or amount) produced by the known agonist alone, full antagonists effect a decrease in PSA levels (or amounts) over the level (or amount) produced by the known agonist, and mixed agonists effect a decrease or inhibition in PSA levels (or amounts) over the level (or amount) produced by the known agonist, the amount of decrease or inhibition is an intrinsic property of the analyte.

The method of the present invention can be used for high throughput screening (HTS) of analytes to identify analytes which are mixed AR agonists or full AR agonists/antagonists. Often chemical entities with useful properties are generated by identifying a chemical compound (called a “lead compound”) with some desirable property or activity, creating variants of the lead compound, and evaluating the property and activity of those variant compounds. The current trend is to shorten the time scale for all aspects of drug discovery, preferably drug discovery directed towards steroid receptors. Because of the ability to test large numbers quickly and efficiently, high throughput screening (HTS) methods are replacing conventional lead compound identification methods.

In one aspect, high throughput screening methods involve providing a library containing a large number of potential AR modulators (candidate compounds). Such “combinatorial chemical libraries” are then screened in one or more assays, to identify those library members particular chemical species or subclasses that display a desired characteristic activity. The compounds thus identified can serve as conventional “lead compounds” or can themselves be used as potential AR modulators.

Devices for the preparation of combinatorial libraries are commercially available (See, for example, 357 MPS, 390 MPS, Advanced Chem Tech, Louisville, Ky.; Symphony, Rainin, Woburn, Ma.; 433A Applied Biosystems, Foster City, Calif.; 9050 Plus, Millipore, Bedford, Ma.). A number of well known robotic systems have also been developed for solution phase chemistries. These systems include automated workstations like the automated synthesis apparatus developed by Takeda Chemical Industries, LTD. (Osaka, Japan) and many robotic systems utilizing robotic arms (Zymate II, Zymark Corporation, Hopkinton, Ma.; Orca, Hewlett-Packard, Palo Alto, Calif.) which mimic the manual synthetic operations performed by a chemist. Any of the above devices are suitable for use with the present invention. The nature and implementation of modifications to these devices (if any) so that they can operate as discussed herein will be apparent to persons skilled in the relevant art. In addition, numerous combinatorial libraries are themselves commercially available (See, for example, ComGenex, Princeton, N.J.; Asinex, Moscow, Russia; Tripos, Inc., St. Louis, Mo.; ChemStar, Ltd, Moscow, Russia; 3D Pharmaceuticals, Exton, Pa.; Martek Biosciences, Columbia, Md.).

Any of the assays described herein are amenable to high throughput screening. As described above, the analytes are preferably screened by the methods disclosed herein. High throughput systems for such screening are well known to those of skill in the art. Thus, for example, U.S. Pat. No. 5,559,410 discloses high throughput screening methods for protein binding, while U.S. Pat. Nos. 5,576,220 and 5,541,061 disclose high throughput methods of screening for ligand/antibody binding.

In addition, high throughput screening systems are commercially available (See, for example, Zymark Corp., Hopkinton, Ma.; Air Technical Industries, Mentor, Ohio; Beckman Instruments, Inc. Fullerton, Calif.; Precision Systems, Inc., Natick, Ma.). These systems typically automate entire procedures including all sample and reagent pipetting, liquid dispensing, timed incubations, and final readings of the microplate in detector(s) appropriate for the assay. These configurable systems provide high throughput and rapid start up as well as a high degree of flexibility and customization. The manufacturers of such systems provide detailed protocols. Thus, for example, Zymark Corp. provides technical bulletins describing screening systems for detecting the modulation of gene transcription, ligand binding, and the like.

The present invention further provides a kit for identifying analytes which are selected from the group consisting of full AR agonists, full AR antagonists, and mixed AR agonist, which comprises providing an expression vector which express an ARLBD and an expression vector which expresses a full-length human AR. Preferably, the kit provides the expression vector which expresses the ARLBD with a human cells which endogenously expresses the full length human AR. Human MDA-MBA453 cells (ATCC HTB-131) are an example of cells which endogenously express the full-length human AR. The ARLBD can also be provided as a cell line which comprises the DNA encoding the ARBD stably integrated into the genome. Preferably, the ARLB is a non-human ARLBD selected from the group consisting of rat ARLBD, mouse ARLBD, and rhesus monkey ARLBD.

In further embodiments, the kit further provides hydroxyapatite for binding full-length AR- or ARLBD-bound analyte. In further still embodiments, the kit provides full agonist controls such as R1881 or DHT or both.

In further still embodiments, the kit further provides LnCap cells such as those which have been deposited as ATCC CRL-1740 or CRL-1740D. In further embodiments still, the kit also includes an ELISA assay for detecting the PSA produced by the LnCap cells.

The present invention further provides methods for identifying agents for treating musculoskeletal disorders such as bone disease(e.g., osteopenia and osteoporosis), bone formation, hematopoietic disorders (e.g., aplastic anemia and myelodysplastic syndromes), neuromuscular disease, neurodegenerative diseases such as Alzheimer's, cognitive impairment, rheumatological disease, wasting disease, obesity, sexual dysfunction, male disorders such as reproductive disorders and primary or secondary male hypogonadism, prostrate cancer, and for hormone replacement therapy (HRT), such as female androgen deficiency which comprises performing the method described above to identify an analyte which is a mixed agonist, such mixed agonists would be expected to be useful for treating at least one of the aforementioned diseases.

All patents, patent applications, government publications, government regulations, and literature references cited in this specification are hereby incorporated herein by reference in their entirety. In case of conflict, the present description, including definitions, will control.

The following examples are intended to promote a further understanding of the present invention.

### EXAMPLE 1

The cell lysates comprising endogenous full-length human AR and the rhARLBD were prepared as follows.

For cell lysates comprising endogenous full-length human AR, MDA-MB-453 cells (ATCC HTB-131) were cultured in culture dishes in complete RPMI 1640 medium (RPMI medium (Gibco 11835-055, Invitrogen, Carlsbad, Calif.) containing 20 mM HEPES, 4 mM L-glutamine, 10 μg/mL human insulin (Calbiochem, San Diego, Calif. 407694-S), 10% fetal bovine serum (FBS), and 20 μg/mL GENTAMICIN (Gibco 15710-072)). Two to three days after seeding the culture dishes and the cells had reached about 70 to 90% confluence, the cells were detached using a standard trypsin method. Cells were collected in complete RPMI 1640 medium and centrifuged at 1000×g for 10 minutes at 4° C. The cell pellet was washed once in PBS and once in TGEM (10 mM Tris-HCl, pH 7.2, 1 mM EDTA, 10% glycerol, 1 mM 2-mercaptoethanol, 10 mM sodium molybdate, and proteinase inhibitor (Roche Molecular Biochemicals (BMB); 1 pellet per 50 or 100 mL buffer) by centrifugation. The cell pellet was then resuspended TGEM at a concentration of about 10⁷ cells/mL, snap frozen in an ethanol-dry ice bath, and stored at −80° C. The frozen cells were thawed in ice water, gently resuspended, and centrifuged at about 10,000×g for 20 minutes at 4° C. The supernatant fraction was used for the binding with the endogenously expressed full-length human AR. MDA-MB453 cells endogenously express about 70,000 AR/cell.

The rhARLBD, which includes amino acids 601 to 895 of the rhesus monkey AR, corresponds to 622 T through 917 Q of the human AR. Cloning the full length rhesus monkey AR from rhesus monkey prostrate mRNA and subsequent subcloning of the rhARLBD was done as described in WO02090529 to Towler and Chen. Briefly, the rhARLBD was PCR amplified from the full rhesus monkey AR and inserted in frame with GST at a SmaI site of the pESP-1 clone. This placed the rhARLBD downstream of the GST-flag tag.

Cell lysates comprising the rhARLBD were prepared as follows. GST-Flag-rhARLBD (Mr=60 kDa) fusion protein was expressed in yeast using the pESP-1 vector (Stratagene, La Jolla, Calif.) according to the manufacturer's protocol. After a sufficient time to express the GST-Flag-rhARLBD the yeast were centrifuged and the yeast pellet resuspended in TGEM/DTT/PI buffer (10 mM Tris-HCl, pH 7.4, 1 mM EDTA, 10% glycerol, 10 mM molybdate, 2 mM dithiothreitol (DTT), 50 μL yeast protease inhibitor cocktail (PI, Sigma Chemicals, St. Louis, Mo.) per gram of yeast and 1/10×^{th} volume of PI complete (PI: Boehringer-Mannheim, Roche Applied Science, Indianapolis, Ind.) per gram of yeast. The yeast cells were lysed by vortexing in the presence of glass beads in cold TGEM/DTT/PI buffer. The cell debris was centrifuged at 10,000×g for 20 minutes at 4° C. The clear supernatant fraction containing the rhARLBD was the lysate used for the assays. Usually, about 25 to 50 μL of lysate as determined by titration was used per assay. In some cases, the rhARLBD fusion protein was purified from the lysate using a batch purification method using anti-Flag M2 affinity gel (Sigma) in TEGM/DTT buffer. The rhARLBD fusion protein was eluted using TEGM/DTT buffer containing 100 μg/mL Flag peptide.

Alternatively, rhARLBD have also been expressed in yeast or BL21 cells as disclosed in commonly owned WO02090529 to Towler and Chen. Receptor lysate can also be prepared as taught for the preparation of the endogenous full-length human AR. If using yeast or bacteria as hosts, the cell suspension is vortexed in-between freezing and thawing.

### EXAMPLE 2

Hydroxyapatite (HAP)-based binding assays were performed as follows: For the hydroxyapatite assays, the amount of lysate used was based on titration using ³H-ligand at concentration about the Kd value of the ligand to the corresponding receptor. In general, the assay was carried at in 150 μL of TEGM buffer. Serial diluted compounds in 25 μL was combined with 25 μL of 120 nM triamcinolone acetonide (TAC, Sigma Chemicals) and about 3 nM ³H-R1881 which had been diluted in 100% ethanol (final ethanol concentration was about 2%. The above mixture is then mixed with about 100 μL of MDA cell lysate or yeast lysate and incubated at 4° C. overnight (about 18 hours). As a further example, about 0.5 nM ³H-R1881+1:5 serial diluted compounds of shown in FIG. 1 (staring concentration at 5 μM and 8 points titration) +50 were added to 100 μL of the MDA or yeast lysate. The second day, 100 μL of 50% (vol/vol) hydroxyapatite (HAP) slurry was added to each sample. The samples were vortexed and incubated on ice for about 10 minutes. The samples in 96-well format were then washed in a solution containing 40 mM Tris, pH 7.5, 100 mM KCl, 1 mM EDTA, and 1 mM EGTA using the FILTERMATE UNIVERSAL HARVESTER (Packard Instrument Co., Meridian, Conn.). The washing process transfers the HAP pellets containing ligand-bound-expressed receptor to UNFILTER-96 GF/B (Packard Instruments Co.) which has been prewetted in 0.5% polyethylimine. The HAP pellets on the filter plates were incubated with 50 μL of MICROSCINT (Packard Instruments Co.) for 2 hours before counted on the TOPCOUNT (Packard Instruments Co.). Results were analyzed using MRLCalc and compared to known ligands, such as 5-α-dihydrotestosterone and unlabeled R1881, which exhibited IC50s of ca. 1 nM. Binding results from MDA endogenously expressed full-length human AR and rhARLBD were compared to identify compounds with >5 fold of right shift in rhARLBD binding. (When using purified GST-Flag rhARLBD, the assay typically contained about 0.25 μg/mL of purified GST-Flag-rhARLBD and 2 nM ³H-R1881 in 100 μL binding reactions containing 50 mM Tris-HCl, pH 7.5, 10% glycerol, 0.8 M NaCl, 1 mg/mL bovine serum albumin (BSA), and 2 mM DTT). The binding reactions were incubated for about 18 hours at 4° C. and process as above).

As shown in FIGS. 2A-D and FIGS. 3A-D, the HAP binding assays using lysates containing rhARLBD or endogenous full-length human AR can classify the compounds into two groups. Group I compounds with <5 fold difference in binding affinity, while Group II has >5 fold difference in binding affinity. PSA assays as in the following example can distinguish if they are agonists, antagonists or mixed agonists. Based on current data, Group I compounds are full agonists or antagonists. Group II compounds appear to be mixed agonist at different degrees. DHT and R1881 are full agonists because they had the same binding affinity for both the full-length human AR and the rhARLBD (FIGS. 2A and 2B, respectively). CASODEX is a full antagonist because it had the same binding affinity to full-length human AR and rhARLBD (FIG. 2C). DHEA is a weak agonist as shown in FIG. 2D. The remainder of the compounds shown in FIG. 1 were determined to be mixed agonists because they had different binding affinities for the full-length human AR and rhARLBD. The rhARLBD caused a significant right-shift in the percent inhibition which was about five-fold.

### EXAMPLE 3

The LnCap PSA assays were performed as follows: For each assay, the wells of a 96 well tissue culture plate were each seeded with about 20,000 LnCap cells/well. For assays in the agonist mode, the test compounds were added after seeding. For antagonist mode assays, the compounds were added 4 to 12 hours after seeding in the presence of 0.5 nM DHT or R1881. PSA levels in the media were measured 24 to 48 hours later using an ELISA kit. For agonist mode, the PSA levels of all tested compounds were compared to that produced by 10 nM of DHT or R1881. For antagonist mode, the percentage of inhibition was calculated by 100×(analyte−0.5 nM DHT or R1881)/0.5 nM of DHT or R1881 of the corresponding PSA levels. Basal level was subtracted from each data.

The LnCap PSA assay in the agonist mode was performed with 10 nM DHT, 10 nM R1881, 1000 nM 4-androstene-3,17β-dione, 1000 nM 4-androstene-3,17β-diol, 1000 nM CPA, 1000 nM compound A, 5000 nM DHEA and 1000 nM CASODEX. The results are presented in FIG. 4A as percent of the activity of 10 nM DHT. As shown, all of the compounds except for CASODEX had agonist activity because they stimulated PSA production in the LnCap cells. The results confirmed that CASODEX is a full antagonist.

The LnCap PSA assay in the antagonist mode was performed with the above compounds at the same concentration using cells which had been seeded in the presence of 0.5 nM DHT or R1881. After measuring PSA levels produced by the LnCap cells in the presence of DHT and R1881, the compounds above were added to the LnCap cells and the PSA levels measured 24 to 48 hours later. The results shown in FIG. 4B show that 4-androstene-3,17β-dione, 4-androstene-3β,17β-diol, CPA, DHEA and compound A are mixed agonists which antagonized the agonist activity of DHT and R1881 to different extents. The results further show that R1881 does not antagonize 0.5 nM DHT activity at 10 nM. Thus, the assay was able to further distinguish full agonists from full antagonists and mixed agonists, and determine relative ratio of agonist to antagonist activity of the mixed agonists.

While the present invention is described herein with reference to illustrated embodiments, it should be understood that the invention is not limited hereto. Those having ordinary skill in the art and access to the teachings herein will recognize additional modifications and embodiments within the scope thereof. Therefore, the present invention is limited only by the claims attached herein.

1. A method for determining whether an analyte has a mixed androgen receptor (AR) agonist activity or an AR activity selected from the group consisting of full AR agonist activity and full AR antagonist activity, which comprises:
(a) providing a full-length human AR and an AR ligand binding domain polypeptide (ARLBD);
(b) mixing the full-length human AR with the analyte and a labeled agonist in a first binding reaction and determining a first 50% inhibitory concentration (IC50) of the analyte for the full-length human AR;
(c) mixing the ARLBD with the analyte and the labeled agonist in a second binding reaction and determining a second IC50 of the analyte for the ARLBD; and
(d) comparing the first IC50 to the second IC50 wherein a first IC50 which is substantially the same as a second IC50 determines that the analyte has the AR activity selected from the group consisting of the full AR agonist activity and the full AR antagonist activity and wherein a second IC50 which is less than the first IC50 determines that the analyte has the mixed AR agonist activity.

2. The method of claim 1 wherein the full-length human AR and the ARLBD are each provided in a cell extract.

3. The method of claim 1 wherein the full-length human AR is provided as an extract from human cells which endogenously express the full-length human AR.

4. The method of claim 3 wherein the human cells are MDA-MB453 cells which have been deposited as ATCC HTB-131.

5. The method of claim 1 wherein the ARLBD is expressed in cells selected from the group consisting of yeast cells, human cells, non-human mammalian cells, insect cells, and bacterial cells.

6. The method of claim 1 wherein the ARLBD is a non-human ARLBD selected from the group consisting of rat ARLBD, mouse ARLBD, and rhesus monkey ARLBD.

7. The method of claim 1 wherein the first and second binding reactions are hydroxyapatite-based displacement reactions.

8. The method of claim 1 wherein the labeled agonist is selected from the group consisting of methyltrienolone (R1881) and 5-α-dihydrotestosterone (DHT).

9. The method of claim 1 wherein the analyte is determined to have the mixed agonist activity when the second IC50 is about five-fold less than the first IC50.

10. The method of claim 1 which further includes a control binding assay wherein the full-length human AR and the ARLBD are each mixed with an agonist or an antagonist and an IC50 is determined for the agonist or the antagonist.

11. The method of claim 10 wherein the agonist is selected from the group consisting of methyltrienolone (R1881) and 5-α-dihydrotestosterone (DHT) and an IC50 is determined for the agonist.

12. The method of claim 1 wherein in step (b) the analyte is labeled and the agonist is unlabeled.

13. A method for determining whether an analyte has an androgen receptor (AR) activity selected from the group consisting of full AR agonist activity, a full AR antagonist activity, and mixed AR agonist activity, which comprises:
(a) providing an analyte which has been determined to have the AR activity selected from the group consisting of full AR agonist activity, full AR antagonist activity, and mixed agonist activity according to the method of claim 1;
(b) providing prostrate tumor cells which produce prostrate-specific tumor antigen (PSA) in the presence of an AR agonist;
(c) adding the prostrate tumor cells to a medium which includes an agonist to provide a first culture of the cells and adding the prostrate tumor cells to a medium without the agonist to provide a second culture of the cells;
(d) adding the analyte of step (a) to the first culture and the second culture and incubating the cells with the analyte for a time sufficient for a control culture of the cells, which includes the agonist and not the analyte, to produce the PSA; and
(e) detecting the amount of PSA produced by the cells in the first culture and the second culture wherein (i) the analyte which stimulates the cells in the second culture to produce the PSA and further stimulates cells in the first culture to produce the PSA determines the analyte to have the full AR agonist activity, (ii) the analyte which does not stimulate the cells in the second culture to produce the PSA and which reduces the amount of the PSA produced by the cells in the first culture determines the analyte to have the full AR antagonist activity, and (iii) the analyte stimulates the cell in the second culture to produce the PSA and antagonizes the ability of the agonist in the first culture to stimulate the production of the PSA determines the analyte to have the mixed agonist activity.

14. The method of claim 13 wherein the prostrate tumor cells are LnCap cells which have been deposited as ATCC CRL-1740 or CRL-1740D.

15. The method of claim 13 wherein the amount of the PSA is detected by ELISA.

16. The method of claim 13 wherein the agonist is selected from the group consisting of methyltrienolone (R1881) and 5-α-dihydrotestosterone (DHT).

17. A method for determining the ratio of agonist to antagonist activity of an analyte having mixed androgen receptor (AR) agonist activity, which comprises:
(a) providing an analyte which has been determined to have the mixed AR agonist activity according to the method of claim 1;
(b) providing prostrate tumor cells which produce prostrate-specific tumor antigen (PSA) in the presence of an AR agonist;
(c) adding the prostrate tumor cells to a medium which includes an agonist to provide a first culture of the cells and adding the prostrate tumor cells to a medium without the agonist to provide a second culture of the cells;
(d) adding the analyte of step (a) to the first culture and the second culture and incubating the cells with the analyte for a time sufficient for a control culture of the cells, which includes the agonist and not the analyte, to produce the PSA; and
(e) detecting the amount of the PSA produced by the cells in the second culture and the first culture wherein the amount of PSA produced in the second culture and the amount of PSA produced in the first culture determines the ratio of agonist to antagonist activity of the analyte.

18. The method of claim 17 wherein the prostrate tumor cells are LnCap cells which are ATCC CRL-1740 or CRL-1740D.

19. The method of claim 17 wherein the amount of the PSA is detected by ELISA.

20. The method of claim 16 wherein the agonist is selected from the group consisting of methyltrienolone (R1881) and 5-α-dihydrotestosterone (DHT).

21. A method for identifying an analyte which has mixed androgen receptor (AR) agonist activity, which comprises:
(a) providing a full-length human AR and a non-human AR ligand binding domain polypeptide (ARLBD);
(b) mixing the full-length human AR with the analyte and a labeled agonist in a first binding reaction and determining a first 50% inhibitory concentration (IC50) of the analyte for the full-length human AR;
(c) mixing the ARLBD with the analyte and the labeled agonist in a second binding reaction and determining a second IC50 of the analyte for the ARLBD; and
(d) comparing the first IC50 to the second IC50 wherein a wherein a second IC50 which is less than the first IC50 identifies the analyte as having the mixed AR agonist activity.

22. The method of claim 21 further including determining the ratio of agonist to antagonist activity of the analyte by
(i) providing prostrate tumor cells which produce prostrate-specific tumor antigen (PSA) in the presence of an AR agonist;
(ii) adding the prostrate tumor cells to a medium which includes an agonist to provide a first culture of the cells and adding the prostrate tumor cells to a medium without the agonist to provide a second culture of the cells;
(iii) adding the analyte to the first culture and the second culture and incubating the cells with the analyte for a time sufficient for a control culture of the cells, which includes the agonist and not the analyte, to produce the PSA; and
(iv) detecting the amount of the PSA produced by the cells in the second culture and the first culture wherein the amount of PSA produced in the second culture and the amount of PSA produced in the first culture determines the ratio of agonist to antagonist activity of the analyte.
